(19) United States
(12) Patent Application Publication
(10) Pub. No.: US 2025/0344992 A1
(43) Pub. Date: Nov. 13, 2025

(54) SMART BIOELECTRONIC PACIFIER FOR REAL-TIME CONTINUOUS MONITORING OF SALIVARY ELECTROLYTES

(71) Applicant: Georgia Tech Research Corporation, Atlanta, GA (US)

(72) Inventor: Woon-Hong Yeo, Atlanta, GA (US)

(21) Appl. No.: 18/861,020

(22) PCT Filed: May 2, 2023
(86) PCT No.: PCT/US2023/066482

§ 371 Date: Oct. 28, 2024

Related U.S. Application Data
(60) Provisional application No. 63/337,328, filed on May 2, 2022.

(51) Int. Cl. A61B 5/00; A61B 5/145; A61B 5/1473; A61B 5/1495

(52) U.S. Cl. CPC A61B 5/4277; A61B 5/14507; A61B 5/14546; A61B 5/1473; A61B 5/1495; A61B 2562/12

(57) ABSTRACT

An exemplary embodiment of the present disclosure provides a device for monitoring salivary electrolytes. The device can include a control circuit, a sensor coupled to the control circuit, and a biocompatible body configured to be inserted into a mouth of a user. The biocompatible body can be configured to house the control circuit and the sensor, and the sensor can be configured to receive saliva from the user and measure an electrolyte level present in the saliva.

### CROSS-REFERENCE TO RELATED APPLICATIONS

This application claims the benefit of U.S. Provisional Application Ser. No. 63/337,328, filed on 2 May 2022, which is incorporated herein by reference in its entirety as if fully set forth below.

### FIELD OF THE DISCLOSURE

The various embodiments of the present disclosure relate generally to medical diagnostic devices, and more particularly to non-invasive medical diagnostic devices for use with neonates.

### BACKGROUND

Over 480,000 μl children, including newborns, receive intensive care each year in the United States. Children admitted to the neonatal intensive care unit (NICU) often require prolonged hospitalization due to their special health care needs caused by premature birth, low birth weight, or health conditions. Continuous monitoring of critical vital signs, such as heart rate (HR), respiration rate (RR), temperature, blood oxygen level (SpO2), blood pressure, and blood ion level, is crucial to preventing deterioration of health conditions and bringing resources in an efficient way for patient care. For example, it is known that the blood sodium level (135-145 mM/L) is related to blood pressure and heart failure, and the blood potassium level (3.6-5.2 mM/L) is associated with stroke. However, existing systems require a bulky, wall-tethered electronic processing unit, including multiple wired electrodes and sensor interfaces attached to the skins with adhesives. Even worse, regular blood testing is required. As a result, these monitoring systems could damage patients' vulnerable skin or induce other serious complications such as thrombus formation, blood vessel occlusion, sepsis, rupture, bleeding, and death.

Monitoring electrolytes is critical for newborns and babies in the intensive care unit. However, the gold standard methods use a blood draw, which is painful and does not offer continuous measurements.

Several studies have demonstrated a positive correlation between blood and salivary ion levels via optical detectors. However, these devices require a rigid, bulky sensing component and additional supporting devices. For example, an integrated circuit, combined either with optical or electrochemical field effect transistor system, has a fragile part from a silicon wafer that could involve harmful health consequences in continuous monitoring. Thus, although, salivary-based detection offers promise as an alternative, existing devices are ineffective for real-time, continuous monitoring of electrolytes due to their rigidity, bulky form factors, and lack of salivary accumulation.

### BRIEF SUMMARY

The present disclosure relates to medical diagnostic devices. An exemplary embodiment of the present disclosure provides a device for monitoring salivary electrolytes. The device can include a control circuit, a sensor coupled to the control circuit, and a biocompatible body configured to be inserted into a mouth of a user. The biocompatible body can be configured to house the control circuit and the sensor, and the sensor can be configured to receive saliva from the user and measure an electrolyte level present in the saliva.

In any of the embodiments disclosed herein, the biocompatible body can include a channel including an inlet configured to receive saliva from the user, a reservoir in fluid communication with the inlet and configured to contain at least a portion of the saliva, and an outlet in fluid communication with the reservoir and configured to eject saliva from the reservoir.

In any of the embodiments disclosed herein, the inlet can include a microfluidic channel in fluid communication with the reservoir, the sensor being disposed in the reservoir.

In any of the embodiments disclosed herein, the biocompatible body can form a pacifier, and the microfluidic channel can be configured to unidirectionally pass saliva from the user's mouth to the reservoir.

In any of the embodiments disclosed herein, the microfluidic channel can include a base layer in which the microfluidic channel is formed and a top layer bonded to the base layer.

In any of the embodiments disclosed herein, the top layer can be bonded to the base layer with a medical grade epoxy.

In any of the embodiments disclosed herein, the base layer and top layer can include a hydrophilic material capable of drawing in the saliva.

In any of the embodiments disclosed herein, the hydrophilic material can include poly(dimethyl siloxane)-poly(ethylene glycol (PDMS-PEG) block copolymer (BCP).

In any of the embodiments disclosed herein, the microfluidic channel can include a depth of between approximately 350-650 micrometers.

In any of the embodiments disclosed herein, the sensor can include a first working electrode and a reference electrode.

In any of the embodiments disclosed herein, the sensor can further include a second working electrode.

In any of the embodiments disclosed herein, the first working electrode, the reference electrode, and the second working electrode can each include a wire-type electrode. The reservoir can include a plurality of upstanding members forming a capillary pattern configured to draw saliva past the first working electrode, the reference electrode, and the second working electrode.

In any of the embodiments disclosed herein, the first working electrode can be configured to detect sodium ions, and the second working electrode can be configured to detect potassium ions.

In any of the embodiments disclosed herein, the first working electrode can include a solid-state electrode, and the second working electrode can include a solid-state electrode.

In any of the embodiments disclosed herein, the first working electrode can further include a composite-coated wire and a sodium selective membrane. The second working electrode can further include a composite-coated wire and a potassium selective membrane.

In any of the embodiments disclosed herein, the control circuit can be configured to obtain, from the sensor, data related to the sodium ions based on potential differences between the first working electrode and the reference electrode, obtain, from the sensor data, related to the potassium ions based on potential differences between the first working electrode and the reference electrode, and transmit the data related to the sodium ions and the data related to the potassium ions to an end-user device.

Another exemplary embodiment of the present disclosure provides a method of manufacturing a pacifier for monitoring salivary electrolytes. The method can include forming a channel, fixing a sensor in the channel, operatively coupling a control circuit to the sensor, and fixing the channel and the control circuit to a pacifier.

In any of the embodiments disclosed herein, the sensor can include a first working electrode and a reference electrode.

In any of the embodiments disclosed herein, the method can further include fixing a second working electrode in the channel and operatively coupling the control circuit to the second working electrode.

In any of the embodiments disclosed herein, forming the channel can include aligning an inlet of the channel with an aperture of the pacifier, forming a reservoir, a microfluidic channel leading from the inlet to the reservoir, and an outlet in a base layer of a material, and bonding a top layer to the base layer with a medical-grade epoxy. Fixing the first working electrode can include placing the first working electrode in the reservoir prior to bonding the top layer to the base layer. Fixing the second working electrode can include placing the second working electrode in the reservoir prior to bonding the top layer to the base layer.

In any of the embodiments disclosed herein, forming the channel can further include placing the medical-grade epoxy on an edge formed where the top layer and the base layer meet such that the edge is hydrophilic.

In any of the embodiments disclosed herein, the material can include PDMS-PEG BCP.

In any of the embodiments disclosed herein, the method can further include sterilizing the pacifier.

In any of the embodiments disclosed herein, the method can further include making the first working electrode, making the second working electrode, and making the reference electrode. Making the first working electrode can include cleansing a first wire coating the cleansed first wire with a composite coating, and coating the composite-coated first wire with a sodium-selective membrane. Making the second working electrode can include cleansing a second wire, coating the cleansed second wire with the composite coating, and coating the composite-coated second wire with a potassium-selective membrane. Making the reference electrode can include cleansing a third wire, coating the cleansed third wire in a resin, and coating the resin-coated third wire in a fluoropolymer-copolymer.

In any of the embodiments disclosed herein, the composite coating can include carbon black suspended in a silicone rubber.

Another exemplary embodiment of the present disclosure provides a method of determining electrolyte levels in a patient. The method can include placing an ion sensing pacifier in a patient's mouth, detecting a concentration of an electrolyte in saliva, and transmitting the concentration to a user.

In any of the embodiments disclosed herein, detecting the concentration of the electrolyte can include continuously drawing saliva from the patient's mouth from an inlet of the ion sensing pacifier through a microfluidic channel to a reservoir, obtaining a first signal from a first working electrode, and comparing the first signal to a reference signal from a reference electrode. The first working electrode and the reference electrode can be disposed in a capillary pattern contained within the reservoir.

In any of the embodiments disclosed herein, the method can further include obtaining a second signal from a second working electrode and comparing the second signal to the reference signal from the reference electrode. The first working electrode and the reference electrode can be disposed in a capillary pattern contained within the reservoir.

In any of the embodiments disclosed herein, comparing the first signal to the reference signal yields a first electrical potential difference, and the method can further include converting the first electrical potential difference to a concentration of a first electrolyte based on a calibration factor.

In any of the embodiments disclosed herein, comparing the second signal to the reference signal yields a second electrical potential difference, and the method can further include converting the second electrical potential difference to a concentration of a second electrolyte based on the calibration factor.

In any of the embodiments disclosed herein, the first working electrode, the reference electrode, and the second working electrode can each include a wire-type electrode. The reservoir can include a plurality of upstanding members forming a capillary pattern configured to draw saliva past the first working electrode, the reference electrode, and the second working electrode.

In any of the embodiments disclosed herein, the first electrolyte can be sodium and the second electrolyte can be potassium.

In any of the embodiments disclosed herein, the first working electrode can be a solid-state electrode and the second working electrode can be a solid-state electrode.

In any of the embodiments disclosed herein, the first working electrode can further include a composite-coated wire and a sodium selective membrane. The second working electrode can further include a composite-coated wire and a potassium selective membrane.

These and other aspects of the present disclosure are described in the Detailed Description below and the accompanying drawings. Other aspects and features of embodiments will become apparent to those of ordinary skill in the art upon reviewing the following description of specific, exemplary embodiments in concert with the drawings. While features of the present disclosure may be discussed relative to certain embodiments and figures, all embodiments of the present disclosure can include one or more of the features discussed herein. Further, while one or more embodiments may be discussed as having certain advantageous features, one or more of such features may also be used with the various embodiments discussed herein. In similar fashion, while exemplary embodiments may be discussed below as device, system, or method embodiments, it is to be understood that such exemplary embodiments can be implemented in various devices, systems, and methods of the present disclosure.

### BRIEF DESCRIPTION OF THE DRAWINGS

The following detailed description of specific embodiments of the disclosure will be better understood when read in conjunction with the appended drawings. For the purpose of illustrating the disclosure, specific embodiments are shown in the drawings. It should be understood, however, that the disclosure is not limited to the precise arrangements and instrumentalities of the embodiments shown in the drawings.

FIG. 1A provides a perspective view of a device for monitoring salivary electrolytes, in accordance with an exemplary embodiment of the present invention.

FIG. 1B provides a perspective view of a device for monitoring salivary electrolytes, in accordance with an exemplary embodiment of the present invention.

FIG. 2A provides a back view of a device for monitoring salivary electrolytes, in accordance with an exemplary embodiment of the present invention.

FIG. 2B provides a back view of a pacifier, in accordance with an exemplary embodiment of the present invention.

FIG. 2C provides an image of the device of FIG. 2A being calibrated, in accordance with an exemplary embodiment of the present invention.

FIG. 3 provides an image of the device of FIG. 1 in a typical use case, in accordance with an exemplary embodiment of the present invention.

FIG. 4A provides a flowchart comparing the device disclosed herein to an existing method, in accordance with an exemplary embodiment of the present invention.

FIG. 4B provides a flowchart comparing the device disclosed herein to an existing method, in accordance with an exemplary embodiment of the present invention.

FIG. 5A provides a plot showing sensor characterization data, in accordance with an exemplary embodiment of the present invention.

FIG. 5B provides a plot showing sensor characterization data, in accordance with an exemplary embodiment of the present invention.

FIG. 6A provides a plot showing sensor characterization data, in accordance with an exemplary embodiment of the present invention.

FIG. 6B provides a plot showing sensor characterization data, in accordance with an exemplary embodiment of the present invention.

FIG. 6C provides a plot showing sensor characterization data, in accordance with an exemplary embodiment of the present invention.

FIG. 7A provides an exploded view of a channel with an integrated sensor, in accordance with an exemplary embodiment of the present invention.

FIG. 7B provides a detail view of the channel of FIG. 7A,

FIG. 8A provides simulation results of fluid transport over time until the channel of FIG. 7A is filled, in accordance with an exemplary embodiment of the present invention.

FIG. 8B provides an experimental demonstration of transporting performance of the channel of FIG. 7A, in accordance with an exemplary embodiment of the present invention.

FIG. 9 provides a flowchart for operation of an electrolyte sensing device, in accordance with an exemplary embodiment of the present invention.

FIG. 10A provides a plot showing a real-time voltage transient for a sodium ion sensor, in accordance with an exemplary embodiment of the present invention.

FIG. 10B provides a plot showing calculated sensitivity of a sodium ion sensor, in accordance with an exemplary embodiment of the present invention.

FIG. 10C provides a plot showing voltage stability test of a sodium ion sensor, in accordance with an exemplary embodiment of the present invention.

FIG. 10D provides a plot showing a real-time voltage transient for a potassium ion sensor, in accordance with an exemplary embodiment of the present invention.

FIG. 10E provides a plot showing calculated sensitivity of a potassium ion sensor, in accordance with an exemplary embodiment of the present invention.

FIG. 10F provides a plot showing voltage stability test of a potassium ion sensor, in accordance with an exemplary embodiment of the present invention.

FIG. 11A provides a plot of water contact angles versus time, in accordance with an exemplary embodiment of the present invention.

FIG. 11B provides a plot of water contact angles versus time, in accordance with an exemplary embodiment of the present invention.

FIG. 12 provides a schematic workflow of a method of manufacturing a channel, in accordance with an exemplary embodiment of the present invention.

FIG. 13A provides a schematic workflow of a method of manufacturing a channel, in accordance with an exemplary embodiment of the present invention.

FIG. 13B provides a schematic workflow of a method of integrating a sensor in a channel, in accordance with an exemplary embodiment of the present invention.

FIG. 14A provides a method of manufacturing a device for sensing electrolytes, in accordance with an exemplary embodiment of the present invention.

FIG. 14B provides a detail view of FIG. 14A.

FIG. 15A provides a cross sectional view of a channel, in accordance with an exemplary embodiment of the present invention.

FIG. 15B provides a cross sectional view of a channel with a sensor integrated therein, in accordance with an exemplary embodiment of the present invention.

FIG. 16 provides a schematic workflow of a method of manufacturing a sensor, in accordance with an exemplary embodiment of the present invention.

FIG. 17A provides a plot showing voltage signals of sodium ion sensor in NaCl solutions, in accordance with an exemplary embodiment of the present invention.

FIG. 17B provides a plot showing voltage signals of sodium ion sensor in NaCl solutions, in accordance with an exemplary embodiment of the present invention.

FIG. 17C provides a plot showing voltage signals of sodium ion sensor in KCl solutions, in accordance with an exemplary embodiment of the present invention.

FIG. 17D provides a plot showing voltage signals of sodium ion sensor in KCl solutions, in accordance with an exemplary embodiment of the present invention.

FIG. 18A provides a flowchart of a method of manufacturing a pacifier for monitoring salivary electrolytes, in accordance with an exemplary embodiment of the present invention.

FIG. 18B provides a flowchart of a method of manufacturing a pacifier for monitoring salivary electrolytes in accordance with an exemplary embodiment of the present invention.

FIG. 19A provides a flowchart of a method of forming a channel, in accordance with an exemplary embodiment of the present invention.

FIG. 19B provides a flowchart of a method of manufacturing a pacifier for monitoring salivary electrolytes in accordance with an exemplary embodiment of the present invention.

FIG. 20A provides a flowchart of a method of making a first working electrode, in accordance with an exemplary embodiment of the present invention.

FIG. 20B provides a flowchart of a method of making a second working electrode, in accordance with an exemplary embodiment of the present invention.

FIG. 20C provides a flowchart of a method of making a reference electrode, in accordance with an exemplary embodiment of the present invention.

FIG. 21A provides a flowchart of a method of determining electrolyte levels in a patient, in accordance with an exemplary embodiment of the present invention.

FIG. 21B provides a flowchart of a method of detecting a concentration of an electrolyte, in accordance with an exemplary embodiment of the present invention.

### DETAILED DESCRIPTION

To facilitate an understanding of the principles and features of the present disclosure, various illustrative embodiments are explained below. The components, steps, and materials described hereinafter as making up various elements of the embodiments disclosed herein are intended to be illustrative and not restrictive. Many suitable components, steps, and materials that would perform the same or similar functions as the components, steps, and materials described herein are intended to be embraced within the scope of the disclosure. Such other components, steps, and materials not described herein can include, but are not limited to, similar components or steps that are developed after development of the embodiments disclosed herein.

The term “microfluidic” as used herein is not intended to limit channels and microchannels of the present disclosure to a particular size, and the microfluidic channels described herein can have many different sizes in accordance with various embodiments of the present disclosure. In some embodiments, the microfluidic channels can have a depth of no more than approximately 1000 microns. In some embodiments, the microfluidic channels can have a depth of no more than approximately 500 microns. In some embodiments, the microfluidic channels can have a depth of between approximately 350 and 500 microns.

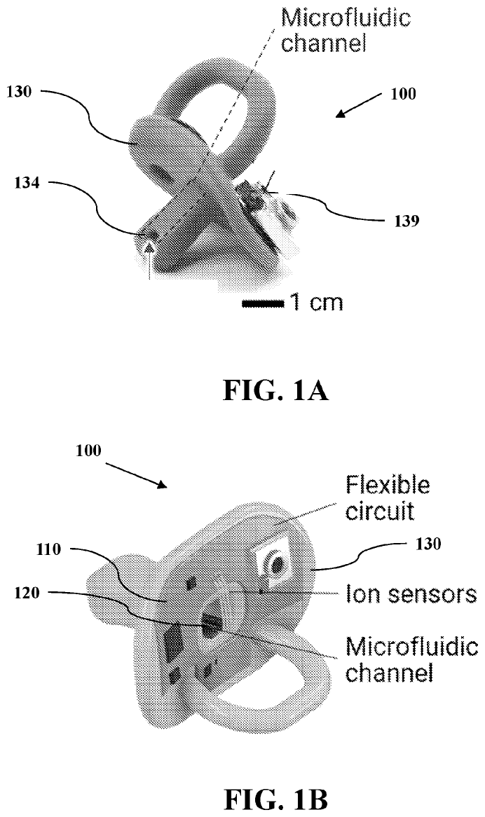

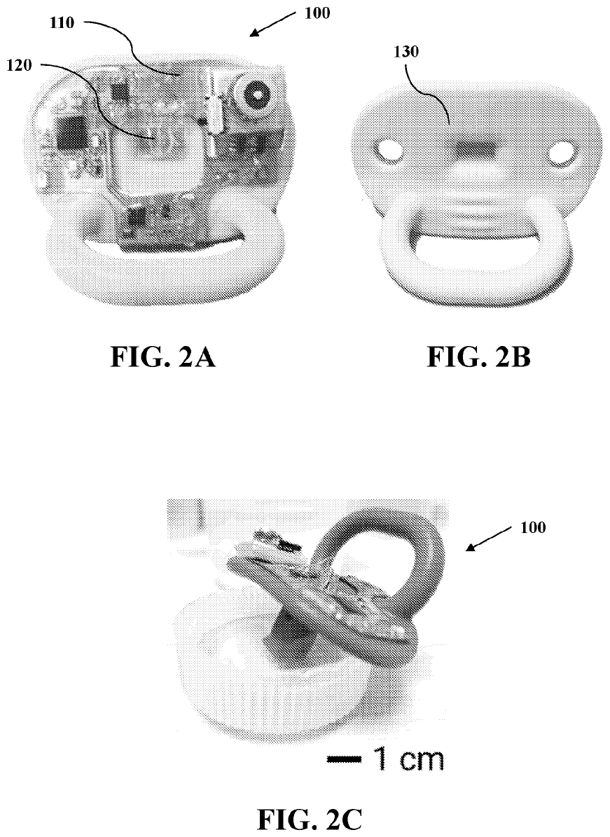

As shown in FIG. 1A, FIG. 1B, and FIG. 2A, an exemplary embodiment of the present disclosure provides a device 100 for monitoring salivary electrolytes. The device 100 can include a control circuit 110, a sensor 120 coupled to the control circuit 110, and a biocompatible body 130 configured to be inserted into a mouth 310 of a user 300. The biocompatible body 130 can be configured to house the control circuit 110 and the sensor 120, and the sensor 120 can be configured to receive saliva from the user 300 and measure an electrolyte level present in the saliva.

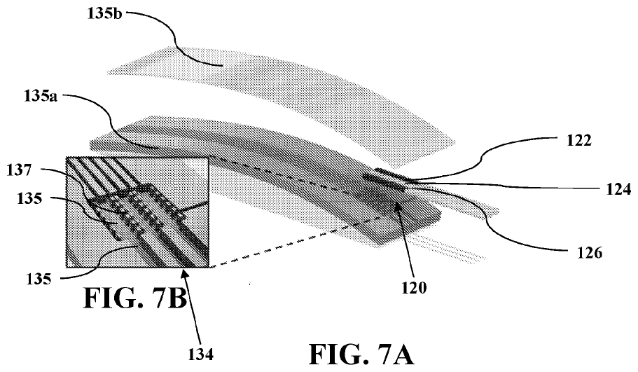

In any of the embodiments disclosed herein, the biocompatible body 130 can include a channel 132 including an inlet 134 configured to receive saliva from the user 300, a reservoir 136 in fluid communication with the inlet 134 and configured to contain at least a portion of the saliva, and an outlet 138 in fluid communication with the reservoir 136 and configured to eject saliva from the reservoir 136. The inlet 134 can include a microfluidic channel 135 in fluid communication with the reservoir 136, the sensor 120 being disposed in the reservoir 136. The channel 132, inlet 134, reservoir 136, and the outlet 138 are shown in more detail in FIGS. 7A-7B, 8A-8B, and 15A-15B.

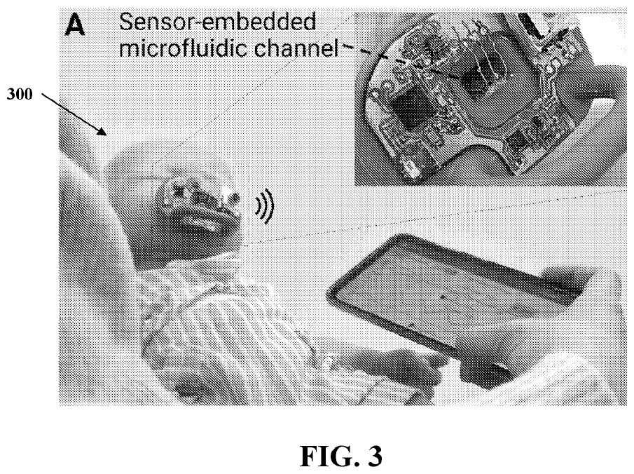

As shown in FIG. 2B, the biocompatible body 130 can form a pacifier, such as a commercially available pacifier, and the microfluidic channel 135 can be configured to unidirectionally pass saliva from the user's mouth to the reservoir 136. Device 100 is shown in the mouth of user 300 and transmitting data to an end user device in FIG. 3.

As shown in FIGS. 7A-7B, the microfluidic channel 135 can include a base layer 135a in which the microfluidic channel 135 is formed and a top layer 135b bonded to the base layer 135a. The top layer 135b can be bonded to the base layer 135a with a medical grade epoxy. The base layer 135a and top layer 135b can include a hydrophilic material capable of drawing in the saliva. The hydrophilic material can include PDMS-PEG BCP.

In some embodiments, the microfluidic channel 135 can include a depth of between approximately 350-650 micrometers.

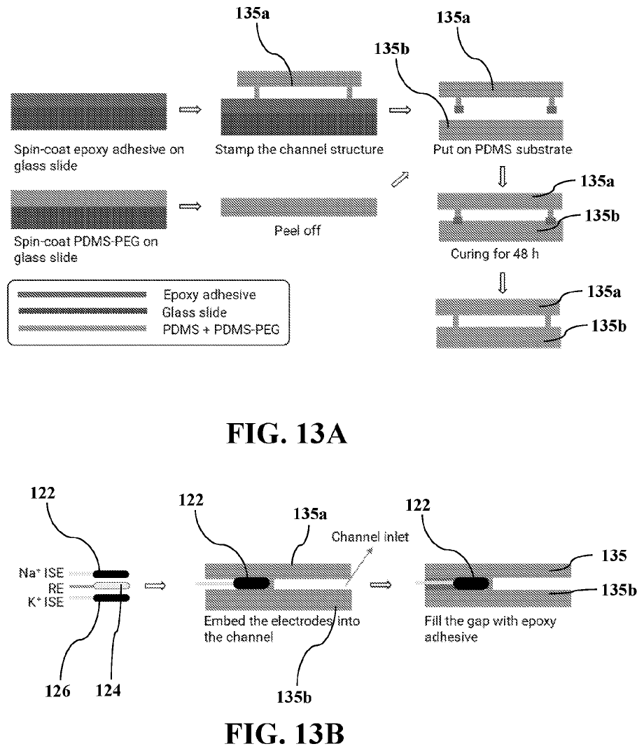

As shown in FIG. 13B, the sensor 120 can include a first working electrode 122 and a reference electrode 124. The sensor 120 can further include a second working electrode 126.

In any of the embodiments disclosed herein, the first working electrode 122, the reference electrode 124, and the second working electrode 126 can each include a wire-type electrode. The reservoir 136 can include a plurality of upstanding members 137 forming a capillary pattern configured to draw saliva past the first working electrode 122, the reference electrode 124, and the second working electrode 126.

In any of the embodiments disclosed herein, the first working electrode 122 can be configured to detect sodium ions, and the second working electrode 126 can be configured to detect potassium ions.

In any of the embodiments disclosed herein, the first working electrode 122 can include a solid-state electrode, and the second working electrode 126 can include a solid-state electrode.

In any of the embodiments disclosed herein, the first working electrode 122 can further include a composite-coated wire and a sodium selective membrane. The second working electrode 126 can further include a composite-coated wire and a potassium selective membrane.

In any of the embodiments disclosed herein, the control circuit 110 can be configured to obtain, from the sensor, data related to the sodium ions based on potential differences between the first working electrode 122 and the reference electrode 124, obtain, from the sensor data, related to the potassium ions based on potential differences between the first working electrode 122 and the reference electrode 124, and transmit the data related to the sodium ions and the data related to the potassium ions to an end-user device.

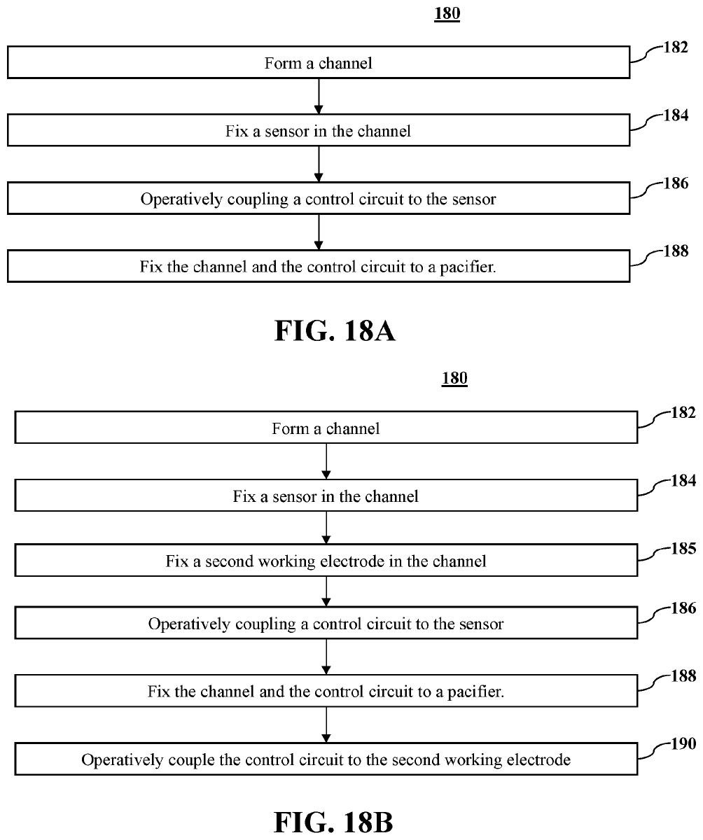

As shown in FIGS. 18A, the present disclosure provides a method 180 of manufacturing a pacifier for monitoring salivary electrolytes. The method 180 can include forming 182 a channel, fixing 184 a sensor in the channel, operatively coupling 186 a control circuit to the sensor, and fixing 188 the channel and the control circuit to a pacifier.

In any of the embodiments disclosed herein, the sensor can include a first working electrode and a reference electrode.

As shown in FIG. 18B, the method 180 can further include fixing 185 a second working electrode in the channel and operatively 190 coupling the control circuit to the second working electrode.

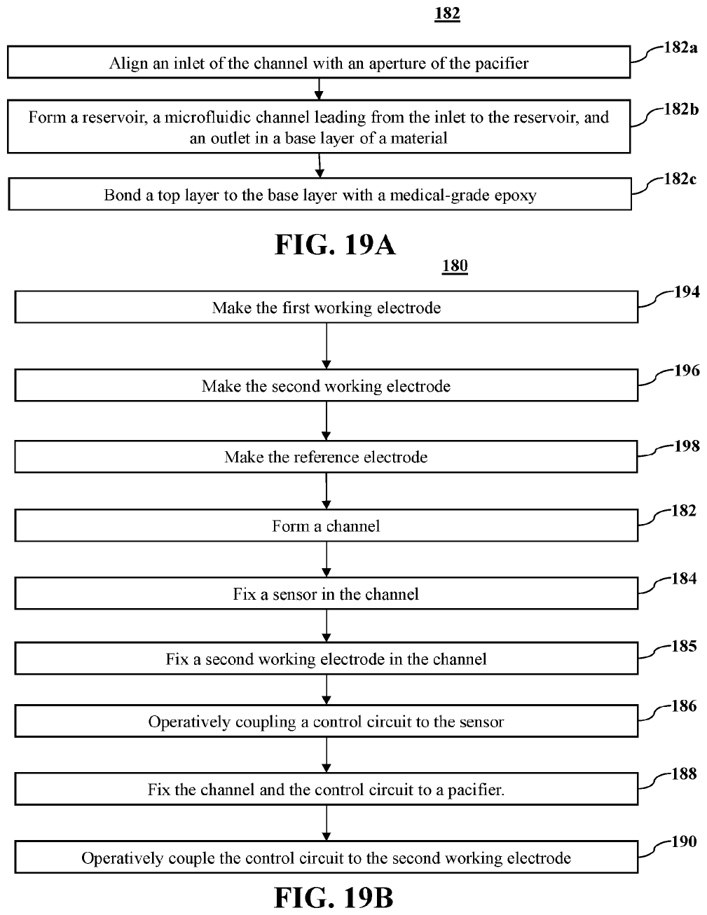

As shown in FIG. 19A, the forming 182 the channel can include aligning 182a an inlet of the channel with an aperture of the pacifier, forming 182b a reservoir, a microfluidic channel leading from the inlet to the reservoir, and an outlet in a base layer of a material, and bonding 182c a top layer to the base layer with a medical-grade epoxy. Fixing the first working electrode can include placing the first working electrode in the reservoir prior to bonding the top layer to the base layer. Fixing 185 the second working electrode can include placing the second working electrode in the reservoir prior to bonding the top layer to the base layer.

In any of the embodiments disclosed herein, forming 182 the channel can further include placing the medical-grade epoxy on an edge formed where the top layer and the base layer meet such that the edge is hydrophilic.

In any of the embodiments disclosed herein, the material can include PDMS-PEG BCP.

In any of the embodiments disclosed herein, the method 180 can further include sterilizing 192 the pacifier.

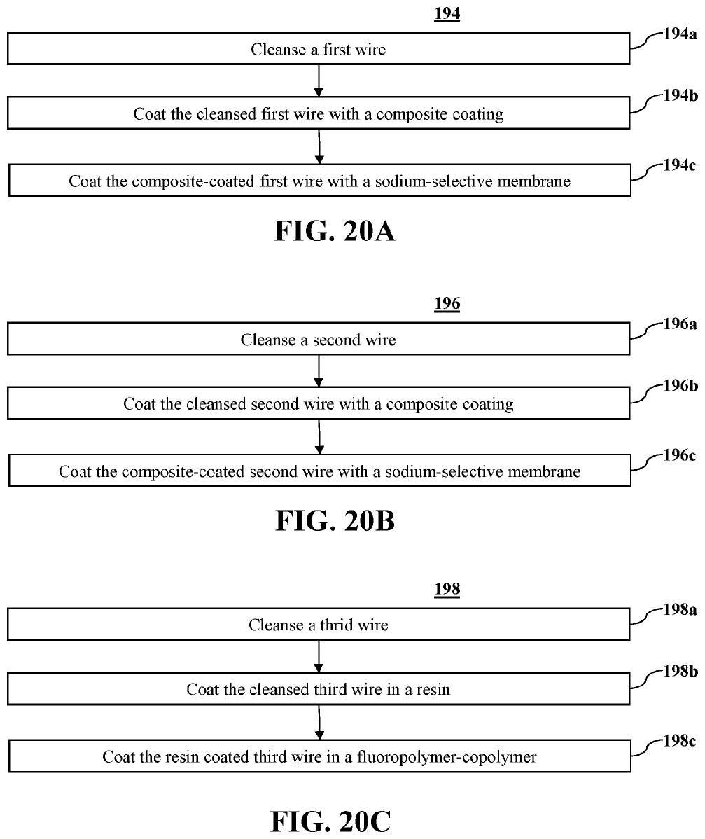

The method 180, as shown in FIG. 19B, can further include making 194 the first working electrode, making 196 the second working electrode, and making 198 the reference electrode. Making 194 the first working electrode, as shown in FIG. 20A, can include cleansing 194a a first wire coating 194b the cleansed first wire with a composite coating, and coating 194c the composite-coated first wire with a sodium-selective membrane. Making 196 the second working electrode, as shown in FIG. 20B, can include cleansing 196a a second wire, coating 196b the cleansed second wire with the composite coating, and coating 196c the composite-coated second wire with a potassium-selective membrane. Making 198 the reference electrode, as shown in FIG. 20C, can include cleansing 198a a third wire, coating 198b the cleansed third wire in a resin, and coating 198c the resin-coated third wire in a fluoropolymer-copolymer.

In any of the embodiments disclosed herein, the composite coating can include carbon black suspended in a silicone rubber.

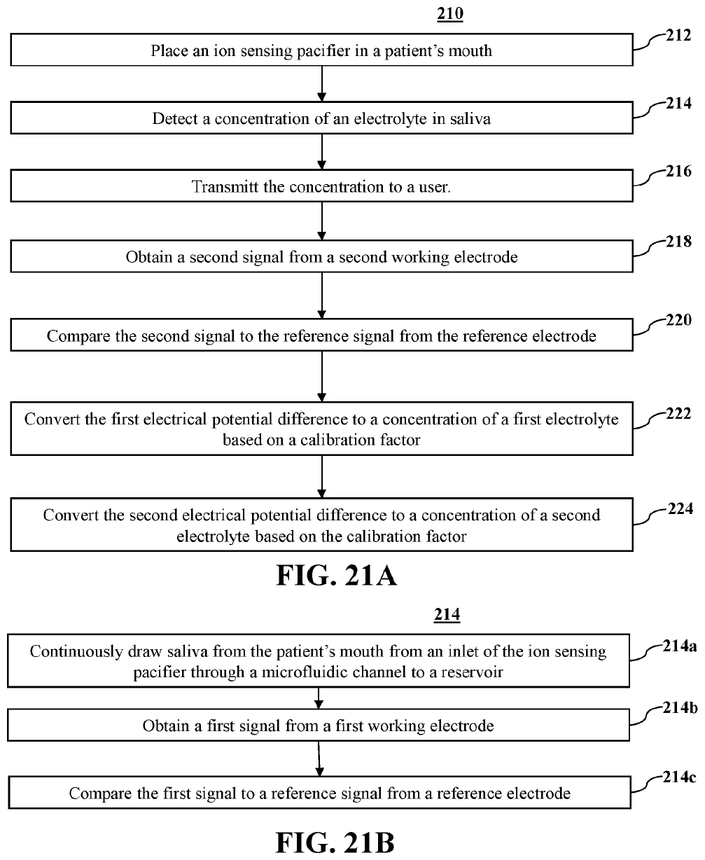

FIG. 21A shows another exemplary embodiment of the present disclosure which provides a method 210 of determining electrolyte levels in a patient. The method 210 can include placing 212 an ion sensing pacifier in a patient's mouth, detecting 214 a concentration of an electrolyte in saliva, and transmitting 216 the concentration to a user.

In the embodiment shown in FIG. 21B, detecting 214 the concentration of the electrolyte can include continuously drawing 214a saliva from the patient's mouth from an inlet of the ion sensing pacifier through a microfluidic channel to a reservoir, obtaining 214b a first signal from a first working electrode, and comparing 214c the first signal to a reference signal from a reference electrode. The first working electrode and the reference electrode can be disposed in a capillary pattern contained within the reservoir.

In any of the embodiments disclosed herein, the method can further include obtaining 218 a second signal from a second working electrode and comparing 220 the second signal to the reference signal from the reference electrode. The first working electrode and the reference electrode can be disposed in a capillary pattern contained within the reservoir.

In any of the embodiments disclosed herein, comparing the first signal to the reference signal yields a first electrical potential difference, and the method 210 can further include converting 222 the first electrical potential difference to a concentration of a first electrolyte based on a calibration factor.

In any of the embodiments disclosed herein, comparing 220 the second signal to the reference signal yields a second electrical potential difference, and the method 210 can further include converting 224 the second electrical potential difference to a concentration of a second electrolyte based on the calibration factor.

In any of the embodiments disclosed herein, the first working electrode, the reference electrode, and the second working electrode can each include a wire-type electrode. The reservoir can include a plurality of upstanding members forming a capillary pattern configured to draw saliva past the first working electrode, the reference electrode, and the second working electrode.

In any of the embodiments disclosed herein, the first electrolyte can be sodium and the second electrolyte can be potassium.

In any of the embodiments disclosed herein, the first working electrode can be a solid-state electrode and the second working electrode can be a solid-state electrode.

In any of the embodiments disclosed herein, the first working electrode can further include a composite-coated wire and a sodium selective membrane. The second working electrode can further include a composite-coated wire and a potassium selective membrane.

The following examples further illustrate aspects of the present disclosure. However, they are in no way a limitation of the teachings or disclosure of the present disclosure as set forth herein.

### Examples

Disclosed herein is an example smart, wireless, bioelectronic pacifier for salivary electrolyte monitoring of neonates, which can detect real-time continuous sodium and potassium levels in real-time without a blood draw. The miniature system facilitates the seamless integration of the ultralight and low-profile device with a pacifier, such as one that is commercially available, without additional fixtures or structural modifications. The portable device includes ion-selective sensors, flexible circuits, and microfluidic channels, allowing simplified measurement protocols in non-invasive electrolyte monitoring. The flexible microfluidic channel enables continuous and efficient saliva collection from a mouth. By modifying the surface properties of the channels and the structure of the capillary reservoir, the device described herein achieve reliable pumping of the viscous medium for quick calibration and measurement. Embedded sensors in the system show good stability and sensitivity: 52 and 57 mV/decade for the sodium and potassium sensor, respectively. In vivo study with neonates in the intensive care unit demonstrates the device's feasibility and performance in saliva-based detection of the critical electrolytes without induced stimulation.

To address the issues of contemporary sensor systems, a miniaturized, potentiometric solid-state ion-selective electrode (SS-ISE) has been adopted as a good solution. The developed SS-ISE successfully replaces fragile components of the conventional electrodes, and allowed for the miniaturization of the sensor's size. However, typical SS-ISEs have an intrinsic instability upon repetitive drying and stretching. Film-type ion sensors also need a relatively wider surface for contacting the analyte for reliable signals. Especially for newborns in the NICU, accuracy is of prime importance to health professionals and caregivers due to the inability of the infants to express their discomfort or illness.

Recently, wearable physiological monitors and glucose detectors have been developed for babies, yet detecting ions still relies on blood measurements. More importantly, studies using SS-ISEs often overlook the correlation between blood and salivary ions, which raises questions about the validity of continuous and non-invasive monitoring. In salivary monitoring, ion levels depend on the sampling methods, sampling sites of interest (i.e., glands), and environmental conditions. For consistent results, it is imperative to standardize methods of supplying fresh saliva to the ion sensor surface.

The device disclosed herein can include a needle-type sensor that can fit into a much narrower space, such as the microfluidic channel structure, than a film-type sensor can. The miniaturized ion sensor, fabricated with thin metal wires, can be embedded in a small inner wall of a commercial pacifier. The overall system is flexible in a small form factor, such that it can be seamlessly attached to a pacifier without additional supporting components or structural modification. The microfluidic channel continuously suctions the saliva from a subject's mouth, enabling real-time monitoring of electrolytes. A specific pattern in the channel maximizes the capillary action against the viscous saliva, securely fixing the sensitive ion sensors in the channel reservoir. Moreover, the microfluidic channel stays hydrophilic at least seven days after oxygen plasma treatment by adding poly(dimethyl siloxane)-poly(ethylene glycol) (PDMS-PEG). The bioelectronic system exploits a low-energy Bluetooth module appropriate for long-term, continuous monitoring of target ions. In vivo study with infants demonstrates the device's performance in continuous salivary electrolyte monitoring from unstimulated saliva. This device can provide evidence for the non-invasive, wireless, continuous, real-time, and easily assessable infant saliva diagnosis.

A fluid simulation was performed with ANSYS FLUENT in order to veriy the flow behavior while the microfluidic channel suctions saliva. For the fluid properties, the viscosity was assumed to 2 cP considering saliva viscosity reported (Male: Mean 1.05, SD 0.42, Female: Mean 1.29, SD 0.70). The density of the saliva was assumed to be the same as water since the composition of the saliva is 99% water. The surface tension of the saliva is 58 mM/N. Those data and assumption indicated that a Reynold number (Re) is much less than one in all channel regions. This means that the flow in the microfluidic channel was laminar throughout the device and viscous effect is dominant. In addition, Weber number (We) was much lower than one, which means that surface tension forces were stronger than inertial forces. Capillary number (Ca) showed the surface forces were also stronger than viscous forces. In summary, the fluid flow physics in the microfluidic channel were governed by viscous and capillary effects.

Thus, the pressure drop in the channel can be expressed as below in Equation 1.

$$Δ p = Q {\frac{12 μ L}{{h}_{0}^{3} w} [ 1 - 0.63 \frac{{h}_{0}}{w} ]}^{- 1} \qquad Equation 1$$

Equation 2 expresses the capillary pressure.

$$Δ p = Q \frac{2 ( h + w )}{hw} σcosθ \qquad Equation 2$$

According to Equation 1 and Equation 2, the microfluidic channel cross sectional decreases upon the pressure drop increases. To avoid the high-pressure drop, a channel width should be larger than 100 μm to avoid the high-pressure drop. The simulation was under laminar flow condition as the Re was calculated to be less than one. The geometry was the same as the final microfluidic channel design. The flow rate was set as inlet velocity (0.0228 mm/s) and the outlet condition was zero-gauge pressure. Based on the microfluidic channel test, the total elapsed time was 25 min. The inlet velocity can be calculated with the volume of the channel chamber and the total elapsed time.

Relating to materials and methods used in these examples: this device can include a Bluetooth-embedded circuit and a sensor-integrated microfluidic channel. The example ion sensors in the system are metal conductors covered with appropriate polymer membranes, all of which are seamlessly integrated with a baby pacifier.

General materials used for devices: Sodium tetrakis-[3,5-bis(trifluoromethyl)phenyl] borate (NaTFPB) was purchased from Alfa Aesar. 4-tert-Butylcalix [4] arene-tetraacetic acid tetraethyl ester (sodium ionophore X), bis(2-ethylhexyl) sebacate (DOS), poly(vinyl chloride) (PVC), tetrahydrofuran (THF), potassium tetrakis(p-chlorophenyl) borate (KTCIPB), hydrochloric acid (HCl), Ag wire, Nafion, Trichloro(1H, 1H,2H,2H-perfluorooctyl) silane and polyvinyl butyral (PVB) were purchased from Sigma Aldrich. Sodium chloride, potassium chloride, calcium chloridedihydrate, and magnesium chloride hexahydrate were from Fisher Chemical. Ecoflex 00-30 was purchased from Smooth-On, and carbon black (CB, Vulcan XC 72R) (CB) was obtained from FuelCellStore. PDMS-PEG BCP (DBE-712) was purchased from Gelest. Medical grade epoxy adhesive was purchased from Epoxy International.

Three ion electrodes were integrated with a flexible circuit. The circuit can include a Bluetooth low-energy chip, a 2.45 GHz chip antenna, and a rechargeable battery 139. The flexible circuit can be used to detect potential differences between the working electrode and the reference electrode. The measured data can be wirelessly transmitted to monitoring devices, such as tablets or smartphones.

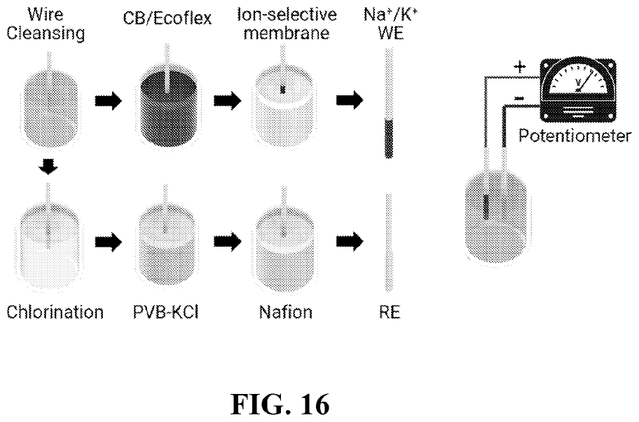

FIG. 16 shows fabrication of electrodes and measurement setup. To make the electrodes, silver wire is sonicated in an IPA bath for 30 min. The wire is cut by 3 centimeters after the cleaning procedure. The CB/Ecoflex composite was prepared by mixing 6 wt % CB and 94% Ecoflex 00-30 in 15 g of toluene by stirring for 30 min at 600 rpm. After the mixing, the composite paste is dip-coated on the pre-cleaned Ag wire. Then, the CB/Ecoflex composite transducer is cured at 150° C. overnight. For the reference electrode, the Ag wire is chlorinated in a 0.1 M KCl and 0.01M HCl solution at 1 mA/cm² for 1 min. 2.5. The CB/Ecoflex electrodes are coated with sodium or potassium ion-selective membranes (ISM) after complete drying. Two types of ISM were used; 1) sodium ISM: sodium ionophore X (2.67 mg), DOS (174.53 mg), PVC (88 mg), NaTFPB (1.47 mg) in 2 mL of THF, and 2) potassium ISM: KTFPB (0.8 mg), Valinomycin (2 mg), PVC (65.8 mg), DOS (131.4 mg) in 2 mL of THF, respectively. The mixtures are vortexed for 6 hours to make a homogeneous solution. The Ag/AgCl RE was coated with a membrane cocktail composed of 78.1 mg PVB, 50 mg KCl, and 1 mL methanol. The resulting ISEs and RE were dried at room temperature overnight.

In order to characterize electrode sensitivity, sodium chloride solutions with different concentrations were used to obtain the sensor information. Considering normal ion levels in human saliva (Sodium: 4 to 37 mM, Potassium: 2.6 to 18.3 mM), solutions with 10⁻³ to 0.1 M were used for repeatability and selectivity. For testing long-term repeatability and selectivity, overnight conditioning was performed and then initiating measurements (when the sensor was fully dried) and at least three times cleaning upon repetitive measurements to remove any residue from the sensor surface. The voltage response of all-solid-state ion ISE and a commercial RE (NT_MRX11) was performed. All sensor measurements were performed with a Gamry potentiostat (Interface 1010E, Gamry Instruments Inc).

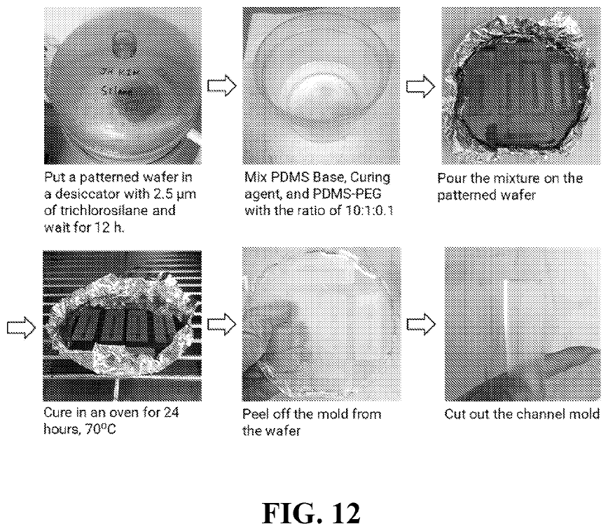

FIG. 12 shows an example fabrication process for a microfluidic channel mold. In order to fabricate the microfluidic channels, PDMS-PEG is exploited as an additive in the hydrophilic modification of the microfluidic channel. PDMS-PEG BCP is added to the PDMS base, and the curing agent mix to obtain 1.0% (w/w). The mixture (PDMS+PDMS-PEG) is blended and poured onto a silicon wafer to cast the microfluidic channel. Trapped air bubbles are removed in a low-pressure desiccator. Before the casting process, the silicon wafer mold should be thoroughly salinized to enhance a clean release process. This process is done by incubating 2 μL droplet of trichlorosilane (Sigma-Aldrich) with the silicon wafer in a low-pressure desiccator overnight. Prepared PDMS-PEG is then poured over the wafer and cured in an oven at 70° C. for 24 hours. Finally, the microfluidic channel structure is removed from the mold, and bonded to a thin PDMS-PEG slab.

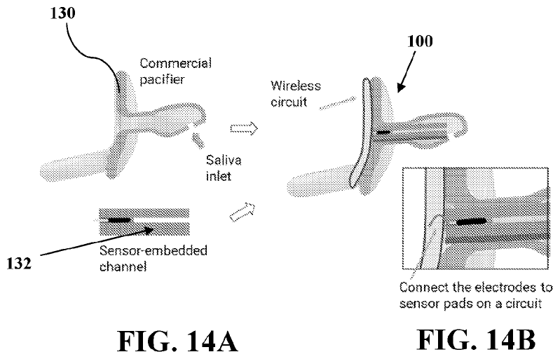

FIGS. 13A-14 show an example integration procedure for a smart pacifier. FIG. 13A shows pin-coating of an epoxy adhesive on a glass slide, and stamping the channel structure and put on the PDMS-PEG spin-coated slide. FIG. 13B shows embedding the ion sensor into the channel. After the integration, the gap iss filled between the channel and the electrodes with epoxy adhesive. FIG. 14 shows attaching the integrated channel structure on the inner wall of the pacifier with medical grade epoxy adhesive. A wireless circuit was mounted on the pacifier with normal epoxy adhesive, while connecting the electrodes to the circuit.

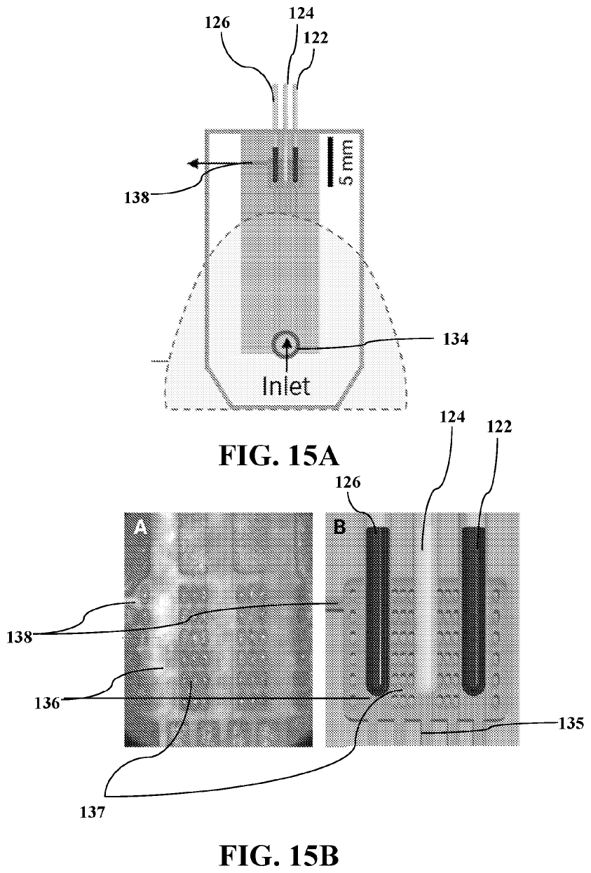

In order to integrate the components of the device, the prepared ion sensors are embedded in the microfluidic channel. The gaps between the ion sensors and the channels are sealed with medical-grade epoxy adhesive and cured at room temperature for 24 hours. A commercially available pacifier sterilized with ethylene oxide (EO) gas to ensure its biosafety is attached to the flexible circuit using medical-grade epoxy adhesive. After curing the epoxy, the microfluidic channel is integrated with ion sensors on the inner wall of the pacifier and connected to the circuit pads via soldering. FIG. 15A shows a cross section of a channel integrated within a pre-sterilized pacifier. The infant holds only a pacifier and a biocompatible inlet while a hydrophilic channel and a capillary reservoir continuously soak saliva samples. FIG. 15B shows the capillary reservoir and ion sensors in microfluidic channels.

Relating to Surface characterization of PDMS-PEG BCP: water droplet contact angles were measured at the polymer sample. A contact angle goniometer (Ossila) was used to obtain the wettability performance of the PDMS-PEG BCP additive. 6 μL of DI water was placed on the PDMS+PDMS-PEG BCP spin-coated glass slide, and the contact angle was measured at 5-min intervals to obtain the timeline data of surface arrangement. Wherever indicated, quantitative data were plotted as the mean±standard deviation (n=3).

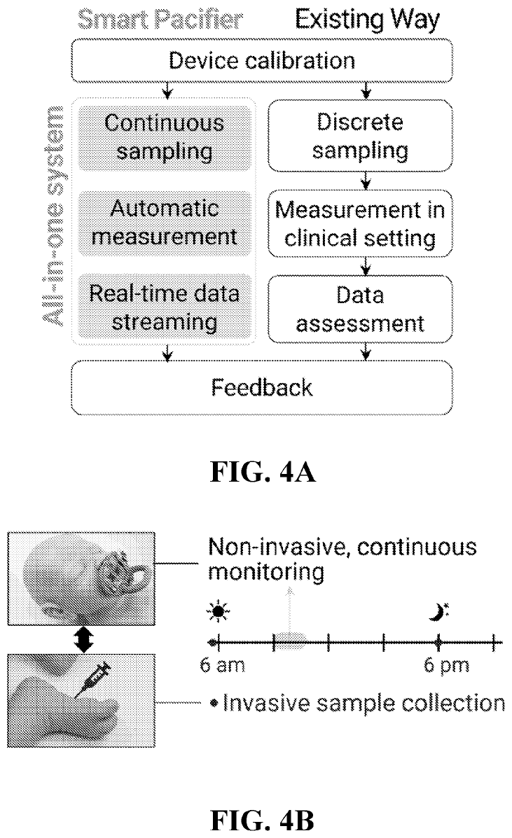

The inventor carried out a clinical study to characterize the example smart pacifier's potential as a wearable non-invasive platform for continuous and real-time monitoring of salivary ions in vivo. The device can include a pacifier, a flexible wireless circuit, a small microfluidic channel embedded with ion sensors, and a rechargeable battery, as shown in FIG. 1A. The flexible circuit can be seamlessly attached to the backside of the pacifier. Another example pacifier is shown in FIG. 1B-2B. Specifically, the channel's end that is exposed at the back of the pacifier soaks up a baby's saliva for continuous flowing through the microchannels. As soon as the pacifier is inserted into the lip, saliva is suctioned through the channels. Then, continuous, and automatic feeding of fresh saliva is followed to a reservoir containing ion sensors. FIGS. 7A-7B, 12, 13A-13B, 14, and 16 show the detailed fabrication process of the example device disclosed herein. Overall, the device assembly follows multiple steps, including fabrication of flexible circuits, ion sensors, and microfluidic channels, integration of the sensors into the microfluidic structure, attachment of the sensor-embedded channel and the circuit to the surface of a pacifier, and final connection of the sensors and a rechargeable battery to the circuit pads via soldering. A rechargeable battery with a magnetic connector can be used. The flow chart in FIG. 4A shows an example method for continuous ion monitoring wirelessly, shown side by side with an example discrete sampling method. The voltage difference between a reference electrode (RE) and two SS-ISEs is measured and recorded by a mobile device with data filtering to suppress random noise signals.

FIGS. 7A-7B describe a layer-by-layer structure of an example embedded microfluidic channel. The channel includes a PDMS-PEG layer, ion sensors, a capillary reservoir, and a PDMS-PEG base layer. The reservoir consists of capillary patterns grouped in multi-lines to fill the gap between the sensors. All designs on the top layers are 500 μm in depth. One of the key advantages of the device disclosed herein is the continuous saliva transportation that obviates the need for conventional discrete and manual sampling. While there have been many prior works regarding PDMS channels, the baby saliva analysis in this work has significant challenges. Saliva is much more viscous than other biofluids such as sweat. The microfluidic channel should be capable of transporting saliva in vertical position. This position is the way the gravity takes effect directly against the capillary effect. The surface of the ion sensors should stay wet during the monitoring. Additionally, PDMS is intrinsically a hydrophobic material that hamstrings the capillary effect. To resolve these challenges, in these examples the inventor use PDMS-PEG and capillary pattern designs.

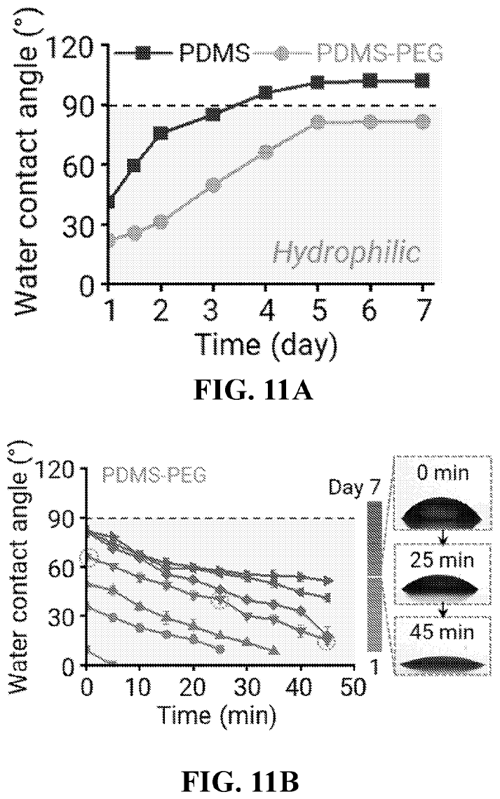

It is well known that PDMS surfaces exposed to oxygen plasma become hydrophilic, and three or four oxygen atoms are bonded to silicon atoms, which reduces hydrophobicity. The main disadvantage of this method is a hydrophobic recovery of PDMS. PDMS-PEG BCP (1% w/w) added when mixing the base and curing agent of PDMS makes PDMS-PEG BCP modified PDMS (PDMS+PDMS-PEG BCP). The PDMS-PEG BCP additive self-assembles at the interface of PDMS to create a hydrophilic PEG layer when exposed to water. The inventor measured sessile drop water contact angles (WCA) to test the hydrophilicity over timescales. The graph in FIG. 11A shows the difference in initial contact angle between PDMS and PDMS-PEG BCP after oxygen plasma treatment. While the WCA of PDMS became above 100°, the WCA of PDMS-PEG BCP remained below 80° even after seven days. Moreover, when it contacts water, PEG group rearrangement renders the surface hydrophilic. FIG. 11B shows decreasing of contact angle over time. When PDMS-PEG BCP was exposed to water, the WCA significantly reduced over time. Higher BCP-containing samples (1.5% and 2.0% PDMS-PEG BCP) showed more hydrophilicity. Nevertheless, the higher percentage of BCP made the samples much viscous before curing. It was too viscous for the trapped bubble to be removed from the samples after the molding step.

Table 1 shows results of water contact angle (WCA) measured on the surface of PDMS-PEG.

TABLE 1

| | | | | | Standard | |
|---|---|---|---|---|---|---|
| | Time | WCA #1 | WCA #2 | WCA #3 | deviation | Average |
| Day | (min) | (°) | (°) | (°) | (°) | WCA (°) |
| 1 | 0 | 8.5 | 7.63 | 11.2 | 1.861532 | 9.11 |
| | 5 | 0 | 0 | 0 | 0 | 0 |
| 2 | 0 | 38.33 | 35.51 | 32.97 | 2.681219 | 35.60333 |
| | 5 | 30.9 | 26.47 | 29.81 | 2.308268 | 29.06 |
| | 10 | 22.32 | 20.61 | 25.48 | 2.470715 | 22.80333 |
| | 15 | 20.11 | 17.63 | 18.9 | 1.240121 | 18.88 |
| | 20 | 16.65 | 13.02 | 17.25 | 2.288733 | 15.64 |
| | 25 | 8.27 | 8.15 | 12.15 | 2.275551 | 9.523333 |
| 3 | 0 | 53.78 | 45.05 | 50.34 | 4.397549 | 49.72333 |
| | 5 | 47.87 | 42.24 | 46.79 | 2.987915 | 45.63333 |
| | 10 | 37.93 | 33.09 | 36.18 | 2.450721 | 35.73333 |
| | 15 | 33.37 | 28.06 | 25.3 | 4.101597 | 28.91 |
| | 20 | 28.25 | 23.75 | 18.87 | 4.691283 | 23.62333 |
| | 25 | 18.99 | 16.67 | 17.68 | 1.163228 | 17.78 |
| | 30 | 13.83 | 15.31 | 12.02 | 1.647756 | 13.72 |
| | 35 | 8.76 | 7.68 | 9.15 | 0.761512 | 8.53 |
| 4 | 0 | 65.21 | 64.48 | 68.9 | 2.369437 | 66.19667 |
| | 5 | 59.59 | 62.58 | 58.91 | 1.952409 | 60.36 |
| | 10 | 51.83 | 51.64 | 56.89 | 2.977756 | 53.45333 |
| | 15 | 46.11 | 49.91 | 50.71 | 2.457641 | 48.91 |
| | 20 | 39.96 | 42.8 | 45.32 | 2.681592 | 42.69333 |
| | 25 | 42.16 | 36.74 | 42.32 | 3.176434 | 40.40667 |
| | 30 | 31.85 | 28.16 | 29.94 | 1.845382 | 29.98333 |
| | 35 | 26.55 | 26.73 | 30.85 | 2.43231 | 28.04333 |
| | 40 | 17.4 | 20.62 | 24.74 | 3.679185 | 20.92 |
| | 45 | 15.25 | 16.35 | 14.15 | 1.1 | 15.25 |
| 5 | 0 | 82.36 | 79.37 | 81.85 | 1.59951 | 81.19333 |
| | 5 | 70.78 | 73.92 | 69.72 | 2.184155 | 71.47333 |
| | 10 | 65.22 | 66.49 | 64.4 | 1.053043 | 65.37 |
| | 15 | 56.95 | 55.67 | 53.6 | 1.690454 | 55.40667 |
| | 20 | 50.92 | 52.85 | 52.43 | 1.015004 | 52.06667 |
| | 25 | 46.31 | 48.11 | 43.86 | 2.133268 | 46.09333 |
| | 30 | 39.24 | 41.24 | 38.83 | 1.289457 | 39.77 |
| | 35 | 40.03 | 35.1 | 36.16 | 2.59504 | 37.09667 |
| | 40 | 31.61 | 34.43 | 33.11 | 1.410957 | 33.05 |
| | 45 | 12.17 | 18.17 | 23.13 | 5.488218 | 17.82333 |
| 6 | 0 | 83.87 | 82.66 | 77.32 | 3.485259 | 81.28333 |
| | 5 | 70.89 | 75.95 | 76.61 | 3.129366 | 74.48333 |
| | 10 | 66.49 | 66.16 | 68.39 | 1.203592 | 67.01333 |
| | 15 | 53.54 | 59.14 | 65.18 | 5.821386 | 59.28667 |
| | 20 | 60.6 | 57.55 | 58.64 | 1.545542 | 58.93 |
| | 25 | 55.23 | 54.78 | 57.75 | 1.600719 | 55.92 |
| | 30 | 53.62 | 50.76 | 55.8 | 2.527634 | 53.39333 |
| | 35 | 48.18 | 49.12 | 52.74 | 2.407682 | 50.01333 |
| | 40 | 42.31 | 46.62 | 44.78 | 2.16266 | 44.57 |
| | 45 | 43.17 | 38.48 | 41.81 | 2.412972 | 41.15333 |
| 7 | 0 | 85.78 | 78.42 | 80.07 | 3.862128 | 81.42333 |
| | 5 | 76.62 | 78.76 | 79.67 | 1.565791 | 78.35 |
| | 10 | 64.96 | 68.93 | 67.29 | 1.994969 | 67.06 |
| | 15 | 60.3 | 64.9 | 62.01 | 2.325088 | 62.40333 |
| | 20 | 59.54 | 59.12 | 60.66 | 0.796074 | 59.77333 |
| | 25 | 56.16 | 60.05 | 56.38 | 2.185154 | 57.53 |
| | 30 | 54.18 | 53.94 | 57.74 | 2.128035 | 55.28667 |
| | 35 | 54.98 | 57.92 | 51.2 | 3.368739 | 54.7 |
| | 40 | 51.97 | 52.75 | 56.67 | 2.518756 | 53.79667 |
| | 45 | 50.02 | 51.02 | 53.21 | 1.631574 | 51.41667 |

Epoxy adhesives are hydrophilic due to polar epoxy groups. For bonding the microfluidic channel, a medical-grade epoxy adhesive can be used. A stamping method can be used to coat the epoxy adhesive on the microfluidic channel. After the slab is put on the microfluidic channel, an epoxy adhesive is squeezed into the microfluidic chamber. Then, the gaps between the slab and the channel are filled to make the edges where the channel and slab chamber meet hydrophilic. The function of the capillary patterns is to enhance the capillary force and prevent the collapse of the reservoir chamber. Next, EO gas is used to sterilize a commercial pacifier to avoid harming baby subjects. Afterward, the sensor-embedded channel and flexible circuit are integrated onto the pre-sterilized pacifier. In this way, a subject is exposed only to a safe portion: a pre-sterilized pacifier and a biocompatible PDMS inlet. Further, the two cm-long channel outlet is positioned at the back of the device, as shown in FIG. 15A so that the used saliva barely reaches and affects the subject after measurement. The channel's ability to soak up fluid allows saliva to pass through in one direction without flowing backward, as shown in FIG. 15A. Ultimately, the example device disclosed herein minimizes toxicity issues associated with sensors, circuits, or batteries through these processes. It also demonstrates the fluid-transporting capability of the microchannel in FIG. 15B with simulation results and experimental validation. The channel successfully transports the saliva vertically even without ion sensors. After the sensor is integrated into the microfluidic channel, the gap between the capillary patterns groups in the reservoir is filled by the ion sensors.

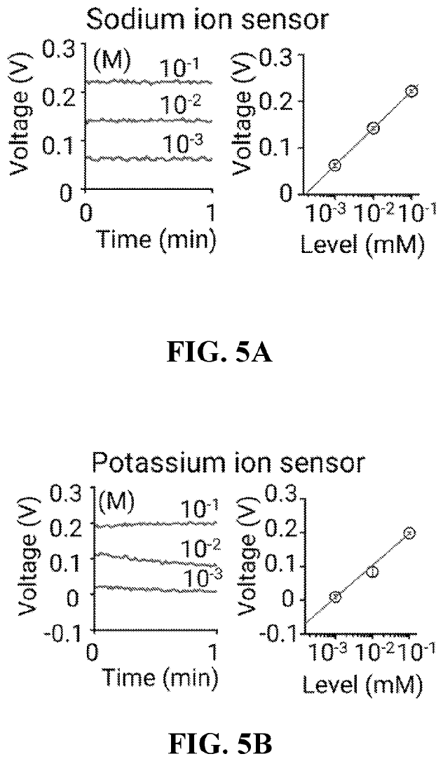

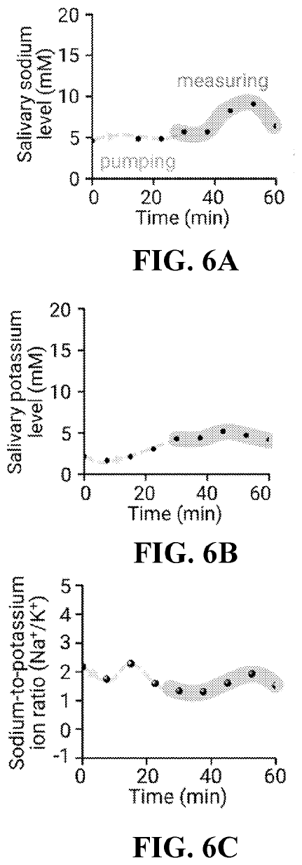

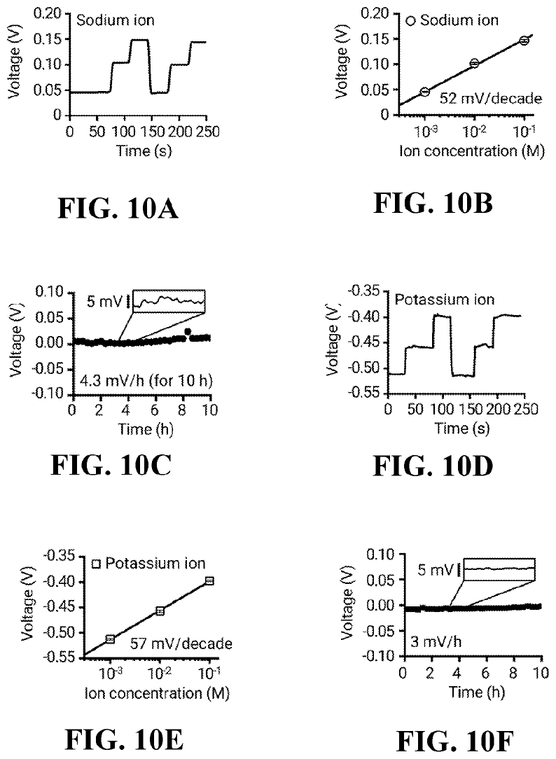

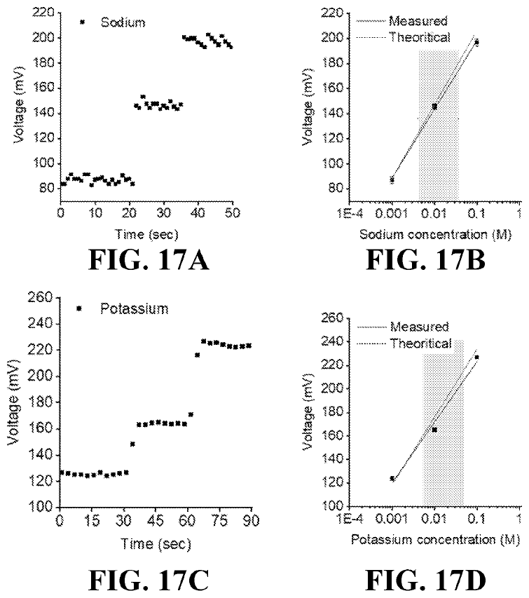

FIGS. 5A-5B and FIGS. 6A-6C summarizes sensors' performance and characterization data. The low-profile, wire-type ion sensors, consisting of a solid-state working electrode (WE) and a reference electrode (RE) can be seamlessly embedded into the prepared microfluidic channels. The detailed fabrication procedure of ion sensors and measurement setup appear in FIG. 16. The WEs of which ion-to-electron transducer is a composite of CB and Ecoflex were coated with sodium and potassium ion-selective membranes. Another silver wire sample was electrochemically chlorinated to coat a chemically stable surface of silver chloride. Subsequently, PVB/KCl and Nafion were coated to negate a dissolution of chloride ions and thus to avoid signal failures. The inventor confirmed the wire-type sensors' functionality. A table-top potentiometer was used to verify the functionality of ion-selective electrodes, compared with the measurement from wireless system shown in FIGS. 17B and 17D and below in Table 2. A real-time voltage transient, measured by the potentiometer, is shown in FIGS. 10A and 10D. The sodium and potassium sensing electrodes show stable, repeatable, and rapid response to 10⁻³, 10⁻², and 10⁻¹ M NaCl and KCl solutions, verifying the good functionality of these wire-type sensors. Their calculated sensitivity (52 mV/decade for sodium; 57 mV/decade for potassium ion sensors) confirms that the electrode response is close to the theoretical values according to the Nernst equation, shown in FIGS. 17B and 17D. The inventor observed similar results when the other edge of the silver wire was soldered to the circuit of the smart pacifier designed to measure and transfer data wirelessly. Voltage stability of sensors is critical to be used in clinical applications; low accuracy of SS-ISE due to an unwanted signal fluctuation is problematic. The inventor resolved the stability issue in this work, as demonstrated in FIGS. 10C and 10F. The electrodes show long-term stability of 4.3 mV/h in NaCl solutions and 3 mV/h in KCl solutions for 10 hours. The inset images in FIGS. 10C and 10F display an enlarged view of the voltage fluctuations for an hour, where standard deviation is 2.0 mV and 0.3 mV for each ion. The demonstrated stability of the sensors is noteworthy considering the device is designed to operate for multiple hours. Furthermore, the results validate the electrodes' structures with particular emphasis on their small wire-like form, which can be easily and seamlessly integrated with miniaturized fluidic channels.

Table 2 shows results of sensing properties measured using a table-top and the wireless devices disclosed herein.

TABLE 2

| | | | | Voltage, | | |
|---|---|---|---|---|---|---|
| Data | | Ion | Voltage, | standard | | Ion levels |
| acquisition | Type of | levels | average | deviation | Sensitivity | deviation |
| unit | ions | (M) | (V) | (V) | (mV/decade) | (mM) |
| Table-top | Sodium | 10⁻³ | 0.045 | ±0.001 | 51.8 (R² = 1) | 0.0 |
| potentiostat | Sodium | 10⁻² | 0.102 | ±0.002 | | 2.0 |
| | Sodium | 10⁻¹ | 0.146 | ±0.002 | | 22.1 |
| | Potassium | 10⁻³ | −0.513 | ±0.002 | 57.4 (R² = 1) | 0.1 |
| | Potassium | 10⁻² | −0.457 | ±0.002 | | 1.6 |
| | Potassium | 10⁻¹ | −0.398 | ±0.002 | | 13.2 |
| Flexible | Sodium | 10⁻³ | 0.087 | ±0.003 | 54.9 (R² = 1) | 0.2 |
| wireless | Sodium | 10⁻² | 0.146 | ±0.002 | | 2.9 |
| circuit | Sodium | 10⁻¹ | 0.197 | ±0.003 | | 26.0 |
| | Potassium | 10⁻³ | 0.124 | ±0.001 | 51.5 (R₂ = 1) | 0.2 |
| | Potassium | 10⁻² | 0.165 | ±0.001 | | 0.7 |
| | Potassium | 10⁻¹ | 0.227 | ±0.001 | | 13.3 |

An example implemented medical use of an example smart pacifier is shown in FIG. 4B, which demonstrates the device's performance by comparing the data with the conventional blood-draw results. For this in vivo study, a commercially available pacifier with integrated sensors and electronics, shown in FIG. 3, was used. After sterilizing the pacifier, the inventor integrated a flexible circuit, sensors, and microfluidic channels with minimal design changes. During the study, only the pre-cleaned area made an oral contact with subjects. In the wearable design of the pacifier, the use of typical pacifiers offers comfort to subjects (babies), while reducing manufacturing costs. FIG. 4B depicts a comparison between non-invasive (smart pacifier) and invasive (blood draw) measurements of ion concentrations in saliva and serum, respectively. The example pacifier disclosed herein provides real-time, continuous monitoring of sodium and potassium ions from saliva for multiple hours. As of now, the electrolyte-based health monitoring in the NICU requires blood draws of babies at least twice daily from their feet, which typically causes bruises. More importantly, blood draw is a single-point, discrete measurement that cannot provide real-time health information of sick babies. There are many types of ion sensors demonstrating great potential for better accuracy and multimodal analysis, but the bulky structures limit to only use spitting saliva. Such system is insufficient to meet the needs for continuous sampling and analysis, especially for babies who cannot collect samples by themselves. Furthermore, the resulting electrolytes' levels are highly dependent on the measurement settings, including sampling sites, methods, and temperatures. These parameters may be neglected in discrete sampling methods. For example, the sodium ion concentrations in saliva vary from 5.6 mM to 70 mM, limiting the clinical use of salivary diagnosis.

A set of data in FIGS. 5A-6C demonstrates unique advantages of the smart pacifier system in detection of sodium and potassium ions. The sensors were calibrated after conditioning using three different calibration solutions. Using a swab and blower, the inventor reduced the calibration time by sucking the remaining solution in the channels. As shown in FIG. 2C, the system is calibrated by using drops with a small volume (<5 mL) of 10⁻³, 10⁻², and 10⁻¹ NaCl and KCl solutions. By leaning the small container approximately 45 degrees, it was possible to capture clear signals due to the microfluidic channels' hydrophilic surfaces. Additionally, the inventor increased the gain of voltage signals for improved measurement accuracy, shown in FIGS. 5A-5B. Using an amplifier-circuit gain, the voltage value and sensitivity are multiplied by a circuit gain that can be adjusted by the user via a mobile app. In this case, when the gain was set to 1.5, the linearities for sodium and potassium ions sensors were 80 and 95 mV/decade, respectively. The sensitivity of these sensors has been calculated at 53 and 63 mV/decade, close to the Nernst level (59 mV/decade). The difference may arise as a result of differences in temperature, voltage stability of the reference electrode, and biofouling on the surface of the sensors. FIG. 6A validates the wearable device's performance in continuous monitoring of salivary sodium and potassium. The measured data shows salivary concentrations of 5.7-9.1 mM for sodium (average value: 7.1 mM) and 4.2-5.2 mM for potassium (average value: 4.6 mM). Note that the low ion levels detected during the first 30 minutes may be attributed to a pumping effect since it can take up to 25 minutes for the viscous fluid to reach the sensors. The ratio of sodium to potassium levels is well known to be related to various health problems such as cardiovascular disease, chronic kidney disease, diabetes, and aldosteronism. As shown in FIG. 6C, the smart pacifier measures the sodium-to-potassium ratio, which can be used to diagnose and prognosis diseases.

In summary, these examples provide insight into the important area of non-invasive salivary monitoring. This approach creates an all-in-one feature that meets the challenges of developing fully automatic non-invasive protocols. It also does not require users to gather a saliva sample-which may require medical personnel if the patients were infants—and to drop the sample on film electrodes in a discrete manner. This wearable device, to provide maximum user comfort and practical efficiency, has been designed to simultaneously implement continuous sampling, auto measurement, and real-time data streaming. To validate the wearable device's accuracy, a correlation with blood ions under discrete sampling conditions in clinical practice is established: y=1.25x+120.7 where x is a sodium level in saliva and y is a sodium level in blood. By using this equation, the blood sodium level is calculated to be 130 mM in average, which is lower than the measured values by the pacifier (139 and 138 mM). The small discrepancy can be explained by the difference between discrete blood draws and continuous salivary detection, which may be affected by temperature, sampling procedures, and gland sites. For example, the well-known Nernst equation indicates that temperature has a significant effect on the resulting potential values. Therefore, in clinical trial (e.g., urinary catheters), the use of temperature sensors is recommended to correct for body temperature differences between the bladder temperature and the equilibration temperature used to prepare the standards. Temperature is an important factor.

This disclosure relates to a portable bioelectronic pacifier system, allowing for a wireless, real-time, continuous detection of sodium and potassium levels without a blood draw. For the first time, the miniaturized wearable system shows a reliable salivary electrolyte monitoring of neonates. In vivo study in the NICU demonstrated the device's capability of monitoring sodium (5.7˜ 9.1 mM) and potassium (4.2˜ 5.2 mM) levels continuously in real-time. The flexible platform, including of a wireless circuit, surface-modified microfluidic channels, and SS-ISEs embedded in a capillary reservoir, together with a pacifier, could offer non-invasive neonatal health monitoring.

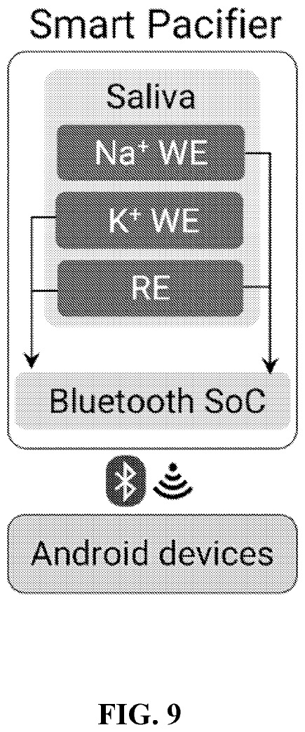

FIG. 9 provides a diagram capturing the key sensing components of the smart pacifier for a wireless data recording with a portable device. FIG. 4A shows a comparison of measurement protocols of the all-in-one ion monitor disclosed herein to existing table-top devices.

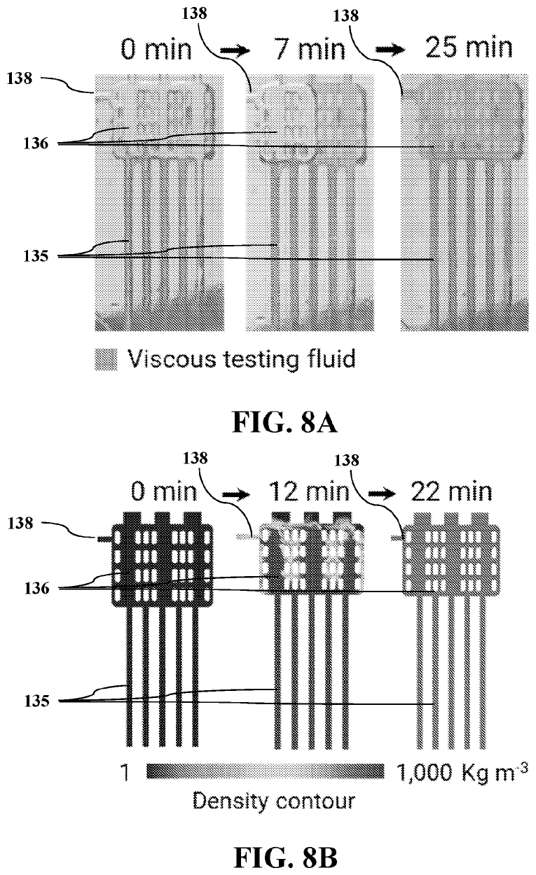

FIG. 11A is a plot showing a comparison of water contact angles on PDMS and PDMS-PEG over time. FIG. 11D show a plot of changes of water contact angles on PDMS-PEG over time. Inset images show photos of contact angles at 0, 25, and 45 minutes on the surface. FIG. 8A shows simulation results of fluid transport over time until the channel is filled. FIG. 8B shows an experimental demonstration of transporting performance of the microfluidic channel, showing a similar trend as estimated in FIG. 8A.

FIGS. 10A-10F relate to the characterization of the ion sensors. FIG. 10A-10B show time-voltage transients measured with different NaCl solution concentrations (10-3, 10-2, and 10-1 M) and the sensitivity of the sodium ion sensor. FIG. 10C shows results from a voltage stability test of a sodium ion sensor for 10 hours along with an enlarged inset for one-hour data. FIG. 10D shows a voltage signal recorded at 10⁻³, 10⁻², and 10⁻¹ M KCl solutions. FIG. 10E shows calculated sensitivity of the potassium sensor. FIG. 10F shows long-term stability test of the sensor for 10 hours (inset: an-hour voltage transients).

FIGS. 17A-17D show voltage signal measured from the wireless circuit and its sensitivity compared to theoretical (Nernst) values. FIGS. 17A-17B show voltage signal of sodium ion sensor at 10-3, 10-2, and 10-1 NaCl solutions. FIGS. 17C-17D show voltage signal of sodium ion sensor at 10-3, 10-2, and 10-1 KCl solutions. The highlighted boxes indicate the known values of sodium and potassium level in saliva.

FIG. 5A shows calibration results of the sensor with sodium ions with different concentrations. FIG. 5B shows calibration results of the sensor for detecting potassium ions. FIG. 6A-6B show sodium and potassium ion levels simultaneously recorded for an hour, demonstrating real-time and continuous monitoring. FIG. 6C shows sodium-to-potassium ion ratio that is useful for prognosis and diagnosis of diseases such as cardiovascular disease risks.

It is to be understood that the embodiments and claims disclosed herein are not limited in their application to the details of construction and arrangement of the components set forth in the description and illustrated in the drawings. Rather, the description and the drawings provide examples of the embodiments envisioned. The embodiments and claims disclosed herein are further capable of other embodiments and of being practiced and carried out in various ways. Also, it is to be understood that the phraseology and terminology employed herein are for the purposes of description and should not be regarded as limiting the claims.

Accordingly, those skilled in the art will appreciate that the conception upon which the application and claims are based may be readily utilized as a basis for the design of other structures, methods, and systems for carrying out the several purposes of the embodiments and claims presented in this application. It is important, therefore, that the claims be regarded as including such equivalent constructions.

Furthermore, the purpose of the foregoing Abstract is to enable the United States Patent and Trademark Office and the public generally, and especially including the practitioners in the art who are not familiar with patent and legal terms or phraseology, to determine quickly from a cursory inspection the nature and essence of the technical disclosure of the application. The Abstract is neither intended to define the claims of the application, nor is it intended to be limiting to the scope of the claims in any way.

1. A device comprising:
a sensor; and
a biocompatible body configured to:
be inserted into a mouth of a user; and
receive saliva from the user and, via a channel, direct saliva into sensory communication with the sensor;
wherein:
the sensor is configured to measure an electrolyte level present in the saliva; and
at least a portion of the channel comprises a hydrophilic material configured to draw saliva into the channel.

2. The device of claim 1, wherein:
the channel comprises a reservoir in which the sensor is disposed; and
at least one of:
the hydrophilic material comprises poly(dimethyl siloxane)-poly(ethylene glycol (PDMS-PEG); or
the sensor comprises a first working electrode, a second working electrode, and a reference electrode, wherein the reservoir comprises upstanding members forming a capillary pattern configured to draw saliva past the first working electrode, the reference electrode, and the second working electrode.

3. (canceled)

4. The device of claim 1, wherein;
the channel comprises:
an inlet comprising a microfluidic channel;
a reservoir in fluid communication with the microfluidic channel and configured to contain:
at least a portion of the saliva; and
the sensor; and
an outlet in fluid communication with the reservoir and configured to exit saliva from the reservoir; and
the microfluidic channel is configured to unidirectionally pass saliva from the user's mouth to the reservoir.

5. The device of claim 4, wherein the channel comprises:
a base layer in which the microfluidic channel is formed; and
a top layer bonded to the base layer.

6. The device of claim 5, wherein the top layer is bonded to the base layer with a medical grade epoxy.

7. The device of claim 5, wherein the base layer and top layer comprise the hydrophilic material.

8. The device of claim 7, wherein the hydrophilic material comprises PDMS-PEG.

9.-11. (canceled)

12. The device of claim 7, wherein:
the sensor comprises:
a first working electrode;
a reference electrode; and
a second working electrode; and
the reservoir comprises upstanding members forming a capillary pattern configured to draw saliva past the first working electrode, the reference electrode, and the second working electrode.

13. The device of claim 7, wherein;
the sensor comprises:
a first working electrode configured to detect sodium ions; and
a second working electrode configured to detect potassium ions.

14. The device of claim 7, wherein:
the sensor comprises:
a first working electrode comprising a solid-state electrode; and
a second working electrode comprising a solid-state electrode.

15. The device of claim 7, wherein;
the sensor comprises:
a first working electrode comprising a composite-coated wire and a sodium selective membrane; and
a second working electrode comprising a composite-coated wire and a potassium selective membrane.

16. The device of claim 13, wherein;
the device further comprises a control circuit;
the sensor further comprises a reference electrode; and
the control circuit is configured to:
obtain, from the sensor, data related to the sodium ions based on potential differences between the first working electrode and the reference electrode;
obtain, from the sensor data, related to the potassium ions based on potential differences between the first working electrode and the reference electrode; and
transmit the data related to the sodium ions and the data related to the potassium ions to an end-user device.

17. A method of manufacturing the device of claim 1 comprising:
forming the channel;
fixing the sensor comprising a first working electrode and a reference electrode in the channel;
operatively coupling a control circuit to the sensor;
fixing the channel and the control circuit to the device.

18. (canceled)

19. The method of claim 17 further comprising:
fixing a second working electrode in the channel; and
operatively coupling the control circuit to the second working electrode.

20. The method of claim 19, wherein forming the channel comprises:
aligning an inlet of the channel with an aperture of the device;
forming a reservoir, a microfluidic channel leading from the inlet to the reservoir, and an outlet in a base layer of a material; and
bonding a top layer to the base layer with a medical-grade epoxy;
wherein fixing the first working electrode comprises placing the first working electrode in the reservoir prior to bonding the top layer to the base layer; and
wherein fixing the second working electrode comprises placing the second working electrode in the reservoir prior to bonding the top layer to the base layer.

21. (canceled)

22. The method of claim 20, wherein the material comprises PDMS-PEG

23.-25. (canceled)

26. A method comprising:
detecting a level of an electrolyte in saliva with the device of claim 1; and
transmitting the level.

27. The method of claim 26, wherein detecting the level of the electrolyte with the device of claim 1 comprises:
continuously drawing saliva from the mouth of the user from an inlet of the channel comprising a microfluidic channel to a reservoir of the channel;
obtaining a first signal from a first working electrode of the sensor; and
comparing the first signal to a reference signal from a reference electrode of the sensor;
wherein the first working electrode and the reference electrode are disposed in a capillary pattern contained within the reservoir.

28. The method of claim 27 further comprising:
obtaining a second signal from a second working electrode of the sensor; and
comparing the second signal to the reference signal from the reference electrode.

29. The method of claim 28, wherein:
comparing the first signal to the reference signal yields a first electrical potential difference;
the method further comprises converting the first electrical potential difference to a concentration of a first electrolyte based on a calibration factor;
comparing the second signal to the reference signal yields a second electrical potential difference; and
the method further comprises converting the second electrical potential difference to a concentration of a second electrolyte based on the calibration factor.

30. (canceled)

31. The method of claim 29, wherein;
the first working electrode, the reference electrode, and the second working electrode each comprise a wire-type electrode;
the reservoir comprises a plurality of upstanding members forming a capillary pattern configured to draw saliva past the first working electrode, the reference electrode, and the second working electrode;
the first electrolyte is sodium and the second electrolyte is potassium;
the first working electrode comprises a solid-state electrode;
the second working electrode comprises a solid-state electrode;
the first working electrode further comprises a composite-coated wire and a sodium selective membrane; and
the second working electrode further comprises a composite-coated wire and a potassium selective membrane.

32.-34. (canceled)
